(19) United States
(12) Patent Application Publication
(10) Pub. No.: US 2006/0111637 A1
(43) Pub. Date: May 25, 2006

(54) Wrist-mount blood pressure monitor with auditory feature

(76) Inventors: Jeffrey M. Jacober, Providence, RI (US); Stephen Lane, Jamestown, RI (US); Aidan Petrie, Jamestown, RI (US); Marco Wo, Providence, RI (US)

Correspondence Address: COOK, ALEX, MCFARRON, MANZO, CUMMINGS & MEHLER LTD, SUITE 2850, 200 WEST ADAMS STREET, CHICAGO, IL, 60606, US

(21) Appl. No.: 11/074,258

(22) Filed: Mar. 7, 2005

Related U.S. Application Data
(63) Continuation-in-part of application No. 10/996,209, filed on Nov. 23, 2004.

(51) Int. Cl. A61B 5/02

(52) U.S. Cl. 600/490; 600/503

(57) ABSTRACT

A wrist-mount blood pressure monitor includes a housing having an anterior leg and a posterior leg rigidly connected together. The anterior leg is opposed and spaced a distance from the posterior leg so that a wrist is allowed to fit in the space between the legs. An inflatable bladder is positioned within the anterior leg of the housing for pressurizing an artery within the wrist. At least one stiffening member can be enclosed within the housing for assuring limited transverse movement between the anterior and posterior legs during pressurization of bladder. A blood pressure monitoring unit is operably connected to the bladder for sensing and calculating blood pressure. An auditory indicator is also provided for audibly outputting instructional information and optionally blood pressure measurement results.

### CROSS REFERENCE TO RELATED APPLICATION

This application is a continuation-in-part of U.S. application Ser. No. 10/996,209 filed on Nov. 23, 2004, now pending and hereby incorporated by reference.

### FIELD OF THE INVENTION

The invention generally relates to wrist-mount blood pressure monitors. More particularly, this invention relates to wrist-mount blood pressure monitors having an improved structure that allows the blood pressure monitor to be conveniently mounted onto a wrist. The improved structure has sufficient rigidness which reduces deformation of the structure during operation and enhances measuring accuracy of the wrist-mount blood pressure monitor. Additionally, the blood pressure monitor includes an auditory feature for informing the user of information related to the use of the blood pressure monitor and optionally related to results of blood pressure measurement.

### BACKGROUND OF INVENTION

Due to increasing health concerns, it has become increasingly important for one to be able to self monitor their blood pressure. Although one may use a blood pressure monitor of the brachial type, a wrist-mount blood pressure monitor is easier and more convenient for at home personal use.

Band-cuff type wrist-mount blood pressure monitors, as shown in U.S. Pat. No. 6,379,310, are well known in the art. In general, blood pressure monitors of this type have a housing or body attached to a cuff. The housing contains a blood pressure monitor unit for sensing and calculating blood pressure, and a display for indicating blood pressure measurement. The cuff of the blood pressure monitor is composed of an inflatable bladder into which fluid, such as air, is supplied. The cuff also includes an elastic cuff band for securing the blood pressure monitor to the wrist. In operation, the blood pressure monitor is positioned on the wrist so that the cuff containing the bladder is placed against the anterior or palm side of the wrist. The elastic band of this type of prior monitor is then wrapped around the wrist and secured to the cuff, usually by a hook and eye fastener. When the blood pressure monitor is activated, the cuff is inflated, pressurizing the anterior of the wrist.

Since the cuff band is made of elastic material, the cuff band stretches and deforms as the cuff is inflated. This results in having to greatly inflate the cuff in order to apply sufficient pressure to the wrist. As the cuff becomes more inflated, the tension generated in the cuff increases. This increase in cuff tension decreases the efficiency of the pressure transmitted from the cuff to an artery within the wrist. The decrease in the efficiency of cuff pressure transmission negatively affects the accuracy of the blood pressure monitor. Most wrist-mount blood pressure monitors measure and calculate blood pressure using the oscillometric method. The oscillometric method is based on the presumption that the pressure of the artery in the wrist is equal to the pressure of the cuff. When the efficiency of pressure transmission is inadequate, the pressure within the cuff becomes higher than the pressure of the artery, and thus the blood pressure monitor inaccurately measures the blood pressure as high. One attempt to solve this problem has been to add a cuff curler to the cuff. The cuff curler is typically made of hard plastic and is placed against the bladder. However, since the blood pressure monitor still employs an elastic band to secure the monitor to the wrist, the above problem still arises.

Another problem that arises with wrist-mount blood pressure monitors that employ a band-cuff is that some people may not have the required level of dexterity to wrap the band around one of their wrists. This problem arises because the hand that corresponds to the wrist to which the blood pressure monitor is mounted is essentially unable to help mount the monitor. Thus, a person mounting the blood pressure monitor to one of his or her wrists has only the use of one hand to maneuver the monitor into the correct position on the wrist, wrap the band snugly around the wrist and secure the band to the cuff. Accomplishing the above mentioned task takes a certain level of dexterity. In general, as people age, their dexterity begins to deteriorate which can make it very difficult for the user to attach a band-cuff type blood pressure monitor to his or her own wrist.

Accordingly, there is a need for a wrist-mount blood pressure monitor which is readily mounted onto the wrist by the person whose blood pressure is to be monitored. Also needed is a self-administered wrist blood pressure monitor that avoids deformation of the structure during inflation of the bladder.

Conventionally, for one to learn how to operate the blood pressure monitor properly, one must be able to read the directions usually contained in an instruction booklet accompanying the blood pressure monitor. However, if a person is blind or has difficulty reading, he or she may not be able to read the instruction booklet easily, increasing the chance of improper usage. The chance of improper usage also increases from one's inexperience in using wrist-mount blood pressure monitors, or one's own forgetfulness on proper usage. Improper usage will most likely result in erroneous blood pressure measurements, thus defeating the purpose of self-monitoring one's own blood pressure.

Previously, some blood pressure monitors have included an auditory feature for relaying usage of the device and results of blood pressure measurement. For example, U.S. Pat. No. 4,558,707, incorporated hereinto by reference, discloses a blood pressure monitor which is said to audibly output the measurements of blood pressure. Further, U.S. Pat. No. 6,800,059, incorporated hereinto by reference, proposes a vital sign box which is said to be capable of measuring many different biological conditions including blood pressure. The vital box is disclosed to be capable of audibly outputting the usage of the vital box and audibly outputting blood pressure measurement.

Accordingly, there is a need for a wrist-mount blood pressure monitor which can be accurately, easily and conveniently mounted onto the wrist without requiring a high level of dexterity. Additionally, there is a need for a blood pressure monitor with the ability to simply and accurately relay information to the user such as directions of use and blood pressure monitor results.

### SUMMARY OF INVENTION

In accordance with the present invention, a wrist-mount blood pressure monitor is provided which has a sufficiently rigid structure that is designed to substantially retain its shape during the operation of the device. The blood pressure monitor also includes a talking or auditory feature for audiably relaying information regarding the blood pressure monitor to the user.

Preferably, the structure has a generally inverted U-shape as used which includes an anterior leg, a posterior leg and a bridge portion connecting the anterior and posterior legs. The anterior and posterior legs are spaced apart a distance to create a gap which allows a wrist to be positioned between the legs.

The blood pressure monitor is preferably mounted onto the left wrist by positioning the anterior leg against the anterior or palm side of the wrist, the posterior leg against the posterior or backhand side of the wrist and the bridge portion against the thumb side of the wrist. An inflatable bladder is located between the anterior leg and the wrist for applying pressure to the wrist.

The anterior leg, posterior leg and bridge portion have substantial rigidity which reduces the amount of transverse movement between the anterior and posterior legs (e.g., elastic deformation) upon inflation of the bladder. A stiffening member may also be employed to aid in limiting transverse movement between the legs. Reducing the amount of elastic deformation allows most of the bladder pressure to be transmitted towards the wrist. This results in requiring less air to inflate the bladder which in turn results in minimizing bladder tension. Thus, the above discussed inaccuracies of measurement and undesirable bladder/cuff tension are substantially avoided.

An additional advantage provided by the structure of the blood pressure monitor of the present invention is that the blood pressure monitor may be conveniently and easily mounted by the person whose blood pressure is to be monitored onto the wrist without requiring a high level of dexterity. A user simply places his or her wrist between the anterior and posterior legs of the device and rests the bridge portion upon the thumb side of the wrist. The blood pressure monitor may also accommodate various wrist sizes such as by providing one or more auxiliary spacing members which may be releaseably attached to the posterior leg.

It is accordingly a general aspect or object of the present invention to provide a wrist-mount blood pressure monitor with an improved structure which is easily mounted onto the user's wrist.

Another aspect or object of this invention is to provide a wrist-mount blood pressure monitor which enhances blood pressure measuring accuracy.

Another aspect or object of this invention is to provide a wrist-mount blood pressure monitor which is conveniently and easily mounted onto a user's wrist without requiring a high level of dexterity.

Another aspect or object of the present invention is to provide an adjustable wrist-mount blood pressure monitor which may be adjustably sized to mount wrists of various sizes.

Another aspect or object of the present invention is to provide a wrist-mount blood pressure monitor with an auditory feature for instructing the user on proper use of the blood pressure monitor.

Another aspect or object of the present invention is to provide a wrist-mount blood pressure monitor with an auditory feature for optionally informing the user of blood pressure monitoring results.

Another aspect or object of the present invention is to provide a wrist-mount blood pressure monitor in which the auditory feature is automatically activated.

Other aspects, objects and advantages of the present invention will be understood from the following description according to the preferred embodiments of the present invention, specifically including stated and unstated combinations of the various features which are described herein, relevant information concerning which is shown in the accompanying drawings.

### BRIEF DESCRIPTION OF THE DRAWINGS

In the course of this description, reference will be made to the accompanying drawings, wherein:

FIG. 1. is a perspective view of an embodiment of a wrist-mount blood pressure monitor of the present invention, shown positioned on a storage/recharger base;

FIG. 2 is a perspective view of the blood pressure monitor shown in FIG. 1;

FIG. 3 is an end elevational view of the blood pressure monitor shown in FIG. 1;

FIG. 4 is an end elevational view of the blood pressure monitor of FIG. 1, shown with one auxiliary spacing member attached to the posterior leg;

FIG. 5 is a perspective view of the blood pressure monitor of FIG. 1, shown positioned upon a user's wrist;

FIG. 6 is an exploded view of the blood pressure monitor of FIG. 1, including an auxiliary spacing member;

FIG. 7 is a perspective view of a blood pressure monitor of the present invention shown with an auditory indicator located in the storage/recharger base; and

FIG. 8 is a perspective view of an alternative embodiment of the blood pressure monitor of FIG. 7, shown with a manual activation switch for activating the auditory indicator.

### DESCRIPTION OF THE PREFERRED EMBODIMENTS

As required, detailed embodiments of the present invention are disclosed herein; however, it is to be understood that the disclosed embodiments are merely exemplary of the invention, which may be embodied in various forms. Therefore, specific details disclosed herein are not to be interpreted as limiting, but merely as a basis for the claims and as a representative basis for teaching one skilled in the art to variously employ the present invention and virtually any appropriate manner.

An embodiment of a wrist-mount blood pressure monitor of the present invention is illustrated in FIG. 1. The blood pressure monitor 10 is shown seated upon a storage base 12. The blood pressure monitor 10 comprises a housing 14 which preferably functions as both a housing for the blood pressure monitor components and a mounting structure for conveniently mounting the blood pressure monitor onto a wrist. The housing 14 preferably has a generally inverted U-shape as used and is made from a rigid material such as polymer resin, metal or the like. For example, ABS resin may be used.

As illustrated in FIGS. 1-4, the housing 14 has an anterior leg 16 which is opposed and spaced a distance from a posterior leg 18. The anterior leg 16 and posterior leg 18 may be connected together at a rigid bridge or connecting portion 20. The bridge portion 20 is preferably arcuate so that the housing has a generally inverted U-shape and generally follows the contour of a typical wrist. The bridge portion may also be non-arcuate or straight so as to form straight angle connections between the anterior and posterior legs, including right angle connections. Additionally, the bridge portion may be either integrally formed with the anterior and posterior legs or a separate connecting member which connects the legs to one another.

As particularly illustrated in FIGS. 3 and 4, a distance “D” is provided between the anterior leg 16 and posterior leg 18. A space or gap 22 is created which is large enough for a wrist to pass through the gap. Preferably, the distance “D” may be adjustably varied between the range of about 1.2 inches (about 3 cm) and about 2 inches (about 5 cm), a range which should accommodate most wrists. This distance can be varied or modified by the use of one or more spacer members discussed in greater detail elsewhere herein.

As illustrated in FIG. 6, the housing is preferably a two-piece housing comprising a top housing 24 and a bottom housing 26 that fit together. The top housing 24 and the bottom housing 26 may be attached to each other by any of the conventional assembly techniques known in the art, such as being snapped together and secured by an adhesive or any variety of fasteners.

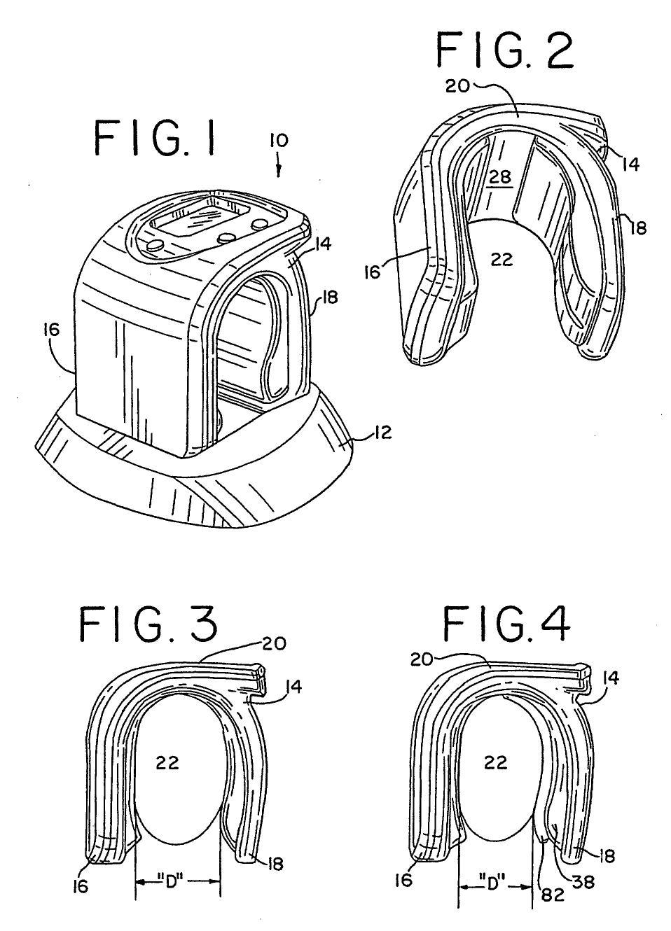

The bottom housing 26 includes an interior wall 28 of the housing 14 (shown in FIG. 2). The interior wall 28 is comprised of an anterior wall 30, which corresponds to the anterior leg, and a posterior wall 32, which corresponds to the posterior leg. A ridge 34, which can serve as a point of attachment with the top housing 24, may project from the periphery of the bottom housing. Additionally, the ridge 34 may also have a rigidness which aids in reducing the transverse movement between the anterior and posterior legs of the housing during operation of the blood pressure monitor.

The anterior wall 30 includes a bladder pressure transmission portion 36 which preferably is comprised of a flexible or pliable material, such as a fabric mesh. The bladder pressure transmission portion 36 may be attached to the bottom housing 26 by heat sealing around the periphery of the portion; however, any other suitable attachment method may be used. The posterior wall 32 preferably includes a pad 38 which may be contoured to the shape of the posterior portion of a wrist. The pad 38 may be integrally formed with the posterior wall 32, separately attached to the posterior wall or positioned against the posterior wall.

Located within the housing in this illustrated embodiment is a tray 40 which preferably comprises a shape similar to the shape of the bottom housing 26. Illustratively, the tray 40 comprises the shape of an inverted “U” when in its in-use orientation as shown in the drawings. When assembled, the tray 40 overlies the bottom housing 26 and is positioned between portions of the ridge 34. The illustrated tray 40 has an inner surface 42 and an outer surface 44. Attached to the inner surface 42 is an inflatable bladder 46 which is positioned between the inner surface 42 of the tray and the anterior wall 30 of the bottom housing, when the blood pressure monitor is assembled. The bladder 46 is positioned so that, as the bladder inflates, bladder pressure may be transmitted through the bladder pressure transmission portion 36 of the anterior wall.

The tray 40 may also provide a place of attachment within the housing for the components of a blood pressure monitoring unit 48, as illustrated. Various blood pressure monitoring units known in the art may be used with the present invention. Such blood pressure monitoring units may include, but are not limited to, the following components: an operation unit having a switch for turning on/off power and starting pressurization of the bladder, a pump for sending air into the bladder, at least one pressure sensor for sensing bladder pressure, a pressure relief valve for venting the bladder, a positioning device and various electronic components needed to operate the blood pressure monitor and calculate blood pressure measurement. Also attached to the tray may be a display unit 50 for displaying blood pressure measurement. When a display unit 50 is used, it is operably connected to the blood pressure monitoring unit 48 and may display diastolic and systolic blood pressure measurement along with pulse rate and mode of operation indicators. The display unit 50 may be read through a display window 52 which is located in the top housing 24. Additionally, instead of a display unit, an auditory indicator, such as a speaker, may be used to notify the user of his or her blood pressure measurement. It is contemplated that the auditory indicator may be located in the base, as describe in more detail below.

It should be understood that it is not a requirement of the present invention that the blood pressure monitoring unit along with any of its various components and the display unit be located within the housing 10. Any or all of these components may be located outside of the housing. Moreover, although there may be production and efficiency reasons for incorporating a member such as a tray 40 for containing and/or mounting various components for operating the unit, other structural and placement members and arrangements are possible or suitable.

The housing 10 may additionally provide a location, such as on the tray 40, for containing batteries 54 which may supply the power needed to operate the blood pressure monitor. Preferably, the blood pressure monitor is designed to operate using either or both rechargeable and/or disposable batteries. The top housing 24 may include a battery hatch or cover 55 as illustrated for providing access to the location of the batteries.

When the blood pressure monitor utilizes rechargeable batteries, the batteries may be recharged by any suitable method known in the art. Preferably, the base 12, of FIG. 1, functions as both a storage and recharging stand. The base 12 may be connected to a household power supply, such as a standard electrical outlet. The top surface of the base 12 may contain recharging plugs and the bottom housing 26 may contain recharging sockets which are adapted to be connected to the recharging plugs. The blood pressure monitor 10 and the base 12 may be constructed so that when the blood pressure monitor is placed upon base 12 for storage, the recharging sockets connect with the recharging plugs allowing the batteries to recharge. When desired, the display unit can provide a battery strength indicator and a recharging status signal.

It should be understood that the batteries could be eliminated from the blood pressure monitor of the present invention and that the blood pressure monitor could be adapted to use household current, using a typical transformer to step down the voltage and electrical wires as needed.

The blood pressure monitor may also include at least one stiffening member which provides a preferred degree of stiffness to the monitor. In the embodiment shown in FIG. 6, the illustrated inverted U-shaped tray 40 has a proximal edge 56 and a distal edge 58. Secured to the proximal edge 56 is a stiffening member 60 and secured to the distal edge is a stiffening member 62. The stiffening members 60,62 preferably comprise a shape similar to the shape of the tray. Illustratively in FIG. 6, the stiffening members have the shape of an inverted “U”. The stiffening members 60, 62 are preferably comprised of a metal such as aluminum or carbon steel; however, the stiffening members may be comprised of any other rigid material. When the tray 40 rests on the bottom housing 26, the stiffening members 60,62 provide sufficient rigidness and support to the anterior leg 16 and posterior leg 18 of the housing 14 so that the distance “D”, shown in FIGS. 3 and 4, between the anterior and posterior legs increases by no more than about three millimeters upon pressurization of the bladder 46 during operation of the blood pressure monitor 10.

It will be understood that the illustrated stiffening members provide an effective and efficient manner to impart the preferred degree of stiffness to the monitor. Other approaches which achieve this objective can be substituted.

The top housing 24 of the illustrated embodiment attaches to bottom housing 26 to enclose the tray 40 and the blood pressure monitoring unit 48 within the housing. The top housing 24 may include openings 64, 66 and 68 which allow access to an on/off switch 70, a memory button 72 and a mode button 74. The top housing may also include a protective cover 75, typically transparent or translucent, for protecting the display unit 50.

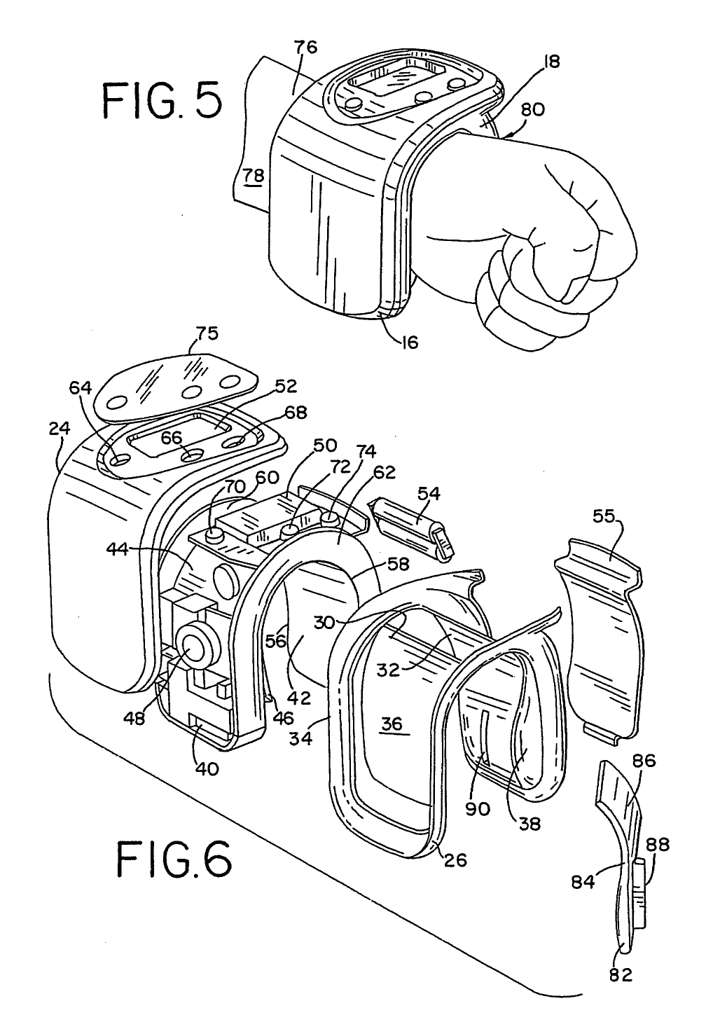

In operation, a user removes the blood pressure monitor from the base 12, when provided. As illustrated in FIG. 5, the blood pressure monitor 10 is preferably mounted onto the left wrist 76 by placing the wrist through the space between the anterior leg 16 and the posterior leg 18 of the blood pressure monitor. The blood pressure monitor 10 is placed on the wrist so that the anterior leg 16 is located on the anterior side of the wrist 78 and the interior wall 30 of the unit, such as its bottom housing 26 in the illustrated embodiment, fits snuggly against the anterior side of the wrist. Additionally, the posterior leg 18 of the housing is located on the posterior side 80 of the wrist so that the posterior wall 32 of the unit, such as pad 38, fits snuggly against the posterior of the wrist.

As seen in FIG. 4, in order to accommodate various wrist sizes, at least one spacer member 82 may be releaseably attached to or positioned over the pad 38 of the posterior leg 18. Turning to FIG. 6, each spacer member 82 includes a front surface 84 and back surface 86. The back surface 86 of this illustrated embodiment includes a projecting member 88 which is adapted to be received into a slot 90 located in the pad 38. The connection between the projecting member 88 and the slot 90 releaseably secures the spacer member to the pad. The spacer member may also include a slot located on the front 84 of the spacer member. This slot is adapted to receive a projecting member of another similarly configured spacer member. It should be understood that when the spacer member is to be releaseably attached to the pad, such can be accomplished by any suitable method known in the art.

Comparing FIG. 3 with FIG. 4, the addition of spacer member 82 inside of the posterior leg (and its pad 38 in this embodiment) reduces the space 22 between the anterior leg 16 and the posterior leg 18. As illustrated, by adding or removing one or more spacer members, a user may adjust the transverse cross-section area which can be accommodated between the anterior and posterior legs to tailor the device according to the user's individual wrist size. Preferably, with the use of spacer members, the space “D” between the anterior and posterior legs of the blood pressure may vary from about 1.2 inches (about 3 cm) to about 2.0 inches (about 5 cm).

Once the blood pressure monitor 10 is mounted onto the wrist as described above, the device is activated, such as by having the user engage the on/off switch to activate the blood pressure monitor. The user then positions the blood pressure monitor at the user's heart level. The blood pressure monitor may or may not contain a positioning device, well known in the art, to indicate to the user whether the blood pressure monitor is in the correct position. As the bladder inflates, the bladder pressure is transmitted to an artery within the anterior portion of the wrist through the bladder pressure transmission portion of the anterior wall. During inflation of the bladder, the stiffening members (or their substitute) support the anterior and posterior legs in order to reduce the amount of outward transverse movement due to bladder pressure. The blood pressure monitoring unit senses and calculates blood pressure which is displayed on the display unit in accordance with approaches generally known in the art. Once the bladder has deflated, the user simply removes the blood pressure monitor from his or her wrist.

As previously mentioned above, the blood pressure monitor 10 may include an auditory indicator located in the base 12 of the blood pressure monitor. The auditory indicator may provide an audio output for instructing the user on the use of the blood pressure monitor and optionally for notifying the user of blood pressure monitor measurement. The auditory indicator may also be used for providing audio outputs for other purposes such as encouraging the user to keep blood pressure under control or tips for lowering blood pressure.

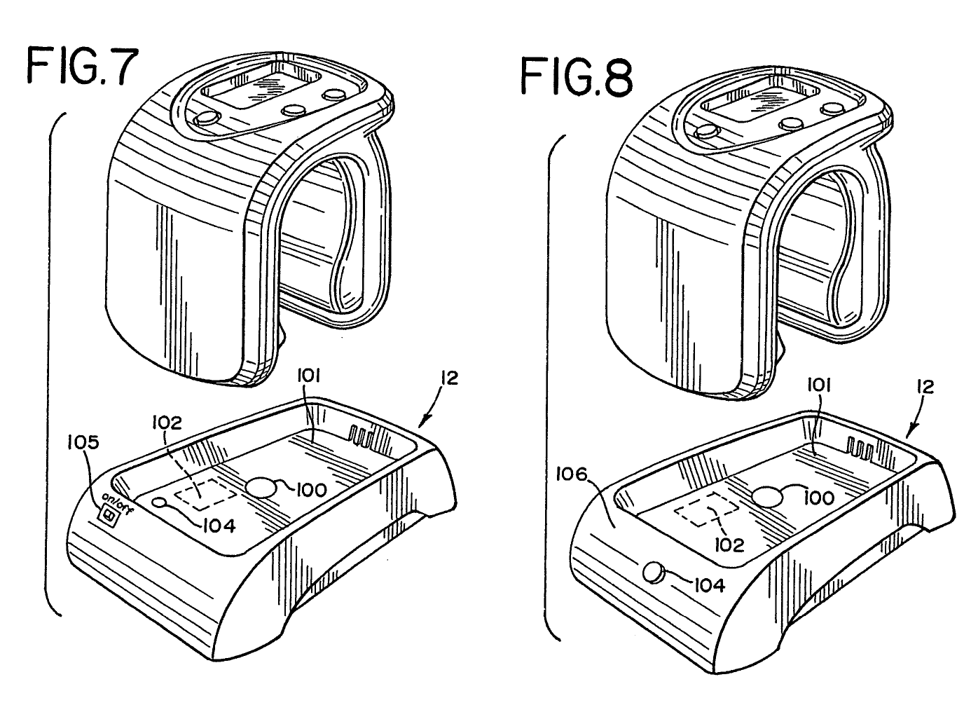

Illustratively, as shown in FIGS. 7 and 8, the auditory indicator may include a speaker 100 located in base 12. The speaker 100 is preferably positioned in the center of top surface 101 the base 12; however, it will be understood that the location of the speaker may not be critical to the over all function of the auditory indicator and the speaker may be placed in another suitable location.

The auditory indicator may also include a sound storage medium such as a microchip 102 (shown in phantom) which contains a set of pre-recorded instructions. The microchip is preferably located within the base and is operably connected to the speaker. In the alternative, the sound storage medium could include a tape recorder instead of an microchip. Additionally, the auditory indicator may be powered by battery or by standard household A/C current.

The auditory indicator may also include a switch 104 which is able to recognize when the blood pressure monitor has been removed from the base. For illustrative purposes, the switch 104 is a pressure sensitive switch which is located on the top surface 101 of the base 12. When the blood pressure monitor 10 is positioned on the base the switch 101 is contacted by the blood pressure monitor and kept in the deactived mode or position. When the monitor 10 is removed from the base the switch 104 is activated, thereby activating the auditory indicator. Additionally, an on/off switch 105 may be incorporated for turning off the auditory indicator feature when it is not desired.

As shown in FIG. 8, the switch 104 may also be a touch button switch located on the outside surface 106 of the base 12 in a position at which a user can simply touch the switch with his or her finger to activate the auditory indicator.

When the auditory indicator is activated, the microchip signals the speaker 100 to output the recorded instructions. The auditory indicator may then output audio information such as the following instructions:

“Welcome to the (name) blood pressure monitor quick start instructions.”
1. Relax and sit comfortably with your feet flat on the floor.
2. Place the blood pressure monitor on your left arm. Position the monitor so that its inflatable bladder is placed on the inside of the arm and the monitor is located ½″ from your wrist bone.
3. Position the monitor at heart level by placing your left hand over your right breast.
4. Press the start button.
5. Stay still while your pressure reading is being taken.
6. When reading is complete record your result, turn the unit off and replace it in the base.

A timer may be incorporated into the auditory indicator so that a predetermined time interval lapses between each outputted instruction. In the alternative, in the embodiment of FIG. 8, the auditory indicator may be configured so that the instructions are outputted step-by-step as the user activates a suitable member, such as by engaging the button 104. It will be understood that the above-recited recorded instructions are provided for illustrative purposes, and that the instructions may be modified as desired.

In operation, the “talking” blood pressure monitor starts out on the storage base 12 as seen in FIG. 1. The user removes the blood pressure monitor from the base. In the embodiment with an automatic switch, as illustrated in FIG. 7, the auditory indicator begins outputting the instructions upon removal of the blood pressure monitor from the base. In the embodiment of FIG. 8, the user must touch the button to activate the auditory indicator. The user then listens to the instructions and operates the blood pressure monitor in accordance with the instructions to measure blood pressure. Once the measurements have been taken, the blood pressure monitor displays the results and/or may be configured to output the results via the auditory indicator. When the user has obtained the results of blood pressure measurement, the user simply returns the blood pressure monitor to the base.

It will be understood that the embodiments of the present invention which have been described are illustrative of some of the applications of the principles of the present invention. Numerous modifications may be made by those skilled in the art without departing from the true spirit and scope of the invention, including those combinations of features that are individually disclosed or claimed herein.

1. A wrist-mount blood pressure monitor comprising:
a housing including an anterior leg and a posterior leg connected to each other, the anterior and posterior legs each including an inner wall, the inner wall of the anterior leg being opposed and spaced a distance from the inner wall of the posterior leg so as to allow access for a wrist to be positioned between the anterior and posterior legs;
an inflatable bladder within the anterior leg of the housing for pressurizing an artery within the wrist;
a portion of the inner wall of the anterior leg adjacent to the bladder comprising a flexible wall so that pressure from the bladder is transmitted to the wrist;
a blood pressure monitoring unit located within the housing; and
an auditory indicator for outputting sound.

2. The blood pressure monitor of claim 1 wherein the auditory indicator includes a speaker and sound storage medium operably connected to the speaker.

3. The blood pressure monitor of claim 1 further comprising a storage base configured to receive and support the blood pressure monitor housing, wherein the auditory indicator is located in the storage base.

4. The blood pressure monitor of claim 2 wherein the sound storage medium comprises a microchip which generates audible information.

5. The blood pressure monitor of claim 3 wherein the base further comprises a switch for activating and deactivating the auditory indicator.

6. The blood pressure monitor of claim 5 wherein the switch automatically actives the auditory indicator upon removal of the blood pressure monitor from the base and automatically deactivates the auditory indicator upon placing the blood pressure monitor on the base.

7. A wrist-mount blood pressure monitor comprising:
an anterior leg;
a posterior leg opposite and spaced a distance from the anterior leg so as to allow access for a wrist to be positioned between the anterior leg and the posterior leg;
a bridge portion that rigidly connects the posterior leg to the anterior leg;
an inflatable bladder positioned between the anterior leg and the wrist for pressurizing an artery within the wrist;
a blood pressure monitoring unit; and
an auditory indicator for outputting sound.

8. The blood pressure monitor of claim 7 wherein the auditory indicator includes a speaker and a sound storage medium operably connected to the speaker.

9. The blood pressure monitor of claim 7 further comprising a storage base configured to receive and support the blood pressure monitor housing, wherein the auditory indicator is located in the storage base.

10. The blood pressure monitor of claim 8 wherein the sound storage medium comprises a microchip which generates audible information.

11. The blood pressure monitor of claim 9 wherein the base further comprises a switch for activating and deactivating the auditory indicator.

12. The blood pressure monitor of claim 11 wherein the switch automatically activates the auditory indicator upon removal of the blood pressure monitor from the base.

13. A wrist-mount blood pressure monitor comprising:
a bottom housing including an anterior leg and a posterior leg rigidly connected together, the anterior leg and posterior leg opposite each other and spaced apart a distance to form a space which allows access for a wrist to be positioned between the legs;
a tray overlying the bottom housing;
a blood pressure monitoring unit attached to the tray;
an inflatable bladder for pressurizing an artery within the wrist, the bladder positioned between the tray and the anterior leg;
a portion of the anterior leg adjacent the bladder comprising a flexible wall so that bladder pressure is transmitted to an artery of the wrist; and
an auditory indicator for outputting sound.

14. The blood pressure monitor of claim 13 further comprising a storage base and a switch for activating and deactivating the auditory indicator, wherein the base is configured so that the auditory indicator and the switch are located in the base.

15. A wrist cuff blood pressure monitor comprising:
a generally U-shaped bottom housing including an anterior leg and a posterior leg with a generally arcuate connecting portion therebetween, the anterior and posterior legs opposite each other and spaced apart a distance to allow access for a wrist to be positioned between the legs;
a generally U-shaped tray overlying the bottom housing;
at least one stiffening member attached to the tray to limit transverse movement between the anterior and posterior legs of the bottom housing;
a blood pressure monitoring unit associated with the tray;
an inflatable bladder for pressurizing an artery of the wrist, the bladder positioned between the tray and the anterior leg;
a portion of a wall of the anterior leg adjacent the bladder comprising a fabric mesh so that bladder pressure is transmitted to an artery of the wrist; and
an auditory indicator for outputting sound.

16. The blood pressure monitor of claim 15 further comprising a storage base and a switch for activating and deactivating the auditory indicator wherein the base is configured so that the auditory indicator and the switch are located in the base.

17. The blood pressure monitor of claim 16 wherein the switch automatically activates the auditory indicator upon removing the blood pressure monitor from the base and deactivates the auditory indicator upon placing the blood pressure monitor on the base.

18. A wrist-mount blood pressure monitor comprising:
a housing including a top housing and a generally U-shaped bottom housing;
the bottom housing including an anterior leg and a posterior leg with a generally arcuate bridge portion therebetween, the anterior and posterior legs being opposite each other and spaced apart a distance to allow access for a wrist to be positioned between the legs;
a generally U-shaped tray within the housing and overlying the bottom housing;
at least one stiffening member attached to the tray to reduce transverse movement between the anterior and posterior legs;
an inflatable bladder for pressurizing an artery of the wrist, the bladder positioned between the tray and the anterior leg;
a portion of the anterior leg adjacent the bladder comprising a fabric mesh so that bladder pressure is transmitted to an artery of the wrist;
a blood pressure monitoring unit within the housing;
a display unit for indicating blood pressure measurement within the housing;
a power supply within the housing;
a storage base for storing the housing;
an auditory indicator located in the base; and
a switch for activating and deactivating the auditory indicator.

19. A method of measuring blood pressure comprising:
providing a wrist-mount blood pressure monitor including an anterior leg and a posterior leg rigidly connected to each other wherein the anterior leg is opposite and spaced a distance from the posterior leg, an inflatable bladder, a blood pressure monitoring unit, a display unit, a storage base for storing the blood pressure monitor and an auditory indicator for outputting instructional information about blood pressure monitor use;
removing the blood pressure monitor from the storage base and activating the auditory indicator;
listening to the instructional information about blood pressure monitor use;
measuring blood pressure in accordance with outputted instructional information;
obtaining results of blood pressure measurement; and
returning the blood pressure monitor to the base.

20. The method of claim 19 wherein said activating comprises automatically activating the auditory indicator upon removing the blood pressure monitor from the base.

21. The method of claim 19 wherein obtaining the results of blood pressure measurement comprises listening to the auditory indicator.
